(19) United States
(12) Patent Application Publication
(10) Pub. No.: US 2026/0080976 A1
(43) Pub. Date: Mar. 19, 2026

(54) SYSTEM AND METHODS FOR GENERATION OF FRAGMENT MODULE LIBRARIES FOR LEAD OPTIMIZATION OF PHARMACEUTICALLY ACTIVE MOLECULES

(71) Applicant: Purdue Research Foundation, West Lafayette, IN (US)

(72) Inventors: Bo Yang, Lafayette, IN (US); Jianing Li, West Lafayette, IN (US)

(73) Assignee: Purdue Research Foundation, West Lafayette, IN (US)

(21) Appl. No.: 19/326,948

(22) Filed: Sep. 12, 2025

Related U.S. Application Data
(60) Provisional application No. 63/694,446, filed on Sep. 13, 2024.

(51) Int. Cl. G16B 35/00; G16B 15/30; G16B 40/20

(52) U.S. Cl. CPC G16B 35/00; G16B 15/30; G16B 40/20

(57) ABSTRACT

A fully automated approach to systematic creation of mega libraries of biologically active derivatives of a specified structural parent drug compound with high potentiality. The libraries may be generated using a variety of discrete steps: creation of backbone libraries and peripheral libraries, introduction of chemical mutations, systematic combination of mutated backbone constituents and peripheral constituents, and systematic attachment of formulated modules to the parent compound to create a mega library of potential lead compounds.

### CROSS REFERENCE TO RELATED APPLICATIONS

This application claims priority to U.S. provisional patent application No. 63/694,446, which was filed Sep. 13, 2024, which are hereby incorporated by reference in their entireties.

### TECHNICAL FIELD

The present disclosure relates to generation of fragment module libraries for lead optimization of drug compounds in silico.

### BACKGROUND

New drug discovery is both time-consuming and expensive. The use of computer-aided drug discovery can accelerate many of the processes involved in drug discovery, particularly the early process of lead optimization, which often involves the systematic mutation of known or suspected pharmaceutically active molecules, with the aim of improving salient chemical properties relating to efficacy, absorption, metabolism, excretion, toxicity and tolerability. To optimize a hit compound, large fragment libraries derived from known drug molecules may be used to explore novel chemical space around the given fragment, while also increasing hit rates and the potency of identified compounds during virtual screening. However, in developing optimal software to achieve these aims, it is critical not simply to build large libraries of fragments, but to generate within these libraries derivative molecules with high potentiality for biological activity and high synthesis accessibility.

### SUMMARY

Presently described is a fully automated approach to systematic creation of libraries of biologically active derivatives of a specified structural parent drug compound with high potentiality and high synthesis accessibility. The present approach can be accomplished through A variety of discrete steps: creation of backbone and peripheral libraries, introduction of chemical mutations, systematic combination of mutated backbone constituents and peripheral constituents, and systematic attachment of formulated modules to the parent compound to create a mega library of potential lead compounds.

To that end, in one aspect, at least one non-transitory computer-readable medium is provide that includes instructions that, when executed by at least one processor, are configured to cause the at least one processor to: (1) receive a dataset of pharmaceutically active parent molecules; (2) identify a template constituent from a respective parent molecule at a cleavage site for each of the patent molecules of the dataset; (3) fragment, according to a first set of rules, each of the identified constituents; (4) sort, according to a second set of rules, each of the fragmented constituents into a backbone library or a peripheral library; (5) mutate, according to a third set of rules, each of the sorted backbone and peripheral constituents; (6) systematically mark, according to a fourth set of rules, (a) each mutated backbone constituent with a dummy atom 1 as a parent attachment and a dummy atom 2 as a peripheral enumeration and (b) each mutated peripheral constituent with the dummy atom 2; (7) systematically combine respective marked backbone constituents and peripheral constituents at any respective dummy atom 2, to generate a dataset of fragment modules; and (8) store the dataset as a searchable fragment module library. In one example of step (4), all fragmented constituents are stored to the backbone library and a copy set of peripheral fragments are saved, according to the second set of rules, into the peripheral library such that both the backbone library and the peripheral library contain a respective original and copy set of peripheral fragments.

In one embodiment, the first set of rules for fragmenting each of the isolated constituents can be engendered in a computer-readable instruction that specifies for cleavage all single bonds in any given constituent that result in fragmenting the constituent into a larger and a smaller fragment, in which the smaller fragment (a) contains at least one heavy atom and (b) has a weight of <150 Da. The second set of rules for sorting each of the fragmented constituents into respective backbone libraries and peripheral libraries (or generating a copy set of peripheral fragments saved to a peripheral library) can be engendered in a computer-readable instruction that specifies that a given fragment is identified as a backbone fragment unless (a) the number non-hydrogen atoms is ≤6 and (b) the cleavage site, i.e., the bond broken to create the fragment, is at a carbon atom, in which case the fragment is identified as a peripheral fragment.

In the same or separate embodiment, the third set of rules for mutating each of the sorted backbone constituents and peripheral constituents can be engendered in a computer-readable instruction that specifies (1), for any atom of a given constituent, a mutation may occur only at a carbon; (2), for any aromatic carbon of a given constituent, (a) if the number of hydrogens is 0, then no mutation may occur; (b) if the number of hydrogens is 1, then (i) the carbon may be replaced by a nitrogen atom or (ii) the hydrogen may be substituted with a halogen; (3) for any aliphatic ring carbon, (a) if the number of hydrogens is 0, then no mutation may occur; (b) if the number of hydrogens is one, then (i) the carbon may be replaced by a nitrogen atom or (ii) the hydrogen may be substituted with a halogen; and (c) if the number of hydrogens is 2, then (i) the carbon may be replaced by a nitrogen atom, (ii) the carbon and the two hydrogens may be replaced by an oxygen atom, a sulfur atom, (iii) the carbon and two hydrogens may be replaced by a carbonyl group; or (iv) one of the hydrogens may be substituted with a halogen and (4) for any aliphatic chain carbon, (a) if the number of hydrogens is 0, then no mutation may occur; (b) if the number of hydrogens is one, then (i) the carbon may be replaced by a nitrogen atom or (ii) the hydrogen may be substituted with a halogen; and (c) if the number of hydrogens is 2 or 3, then (i) the carbon may be replaced by a nitrogen, (ii) the carbon and two hydrogens may be replaced by an oxygen atom, a sulfur atom, (iii) the carbon and two hydrogens may be replaced by a carbonyl group; or (iv) one of the hydrogens may be substituted with a halogen. In examples, all possible mutated permutations are generated in accordance with the instructions. In addition to nitrogen, oxygen, and phosphorous, other non-reactive metals may be used to form mutations as appropriate, e.g., phosphorous, selenium.

The fourth set of rules for mutating each of the sorted backbone constituents and peripheral constituents can be engendered in a computer-readable instruction that specifies that (a) for any given mutated backbone constituent, dummy atoms 1 and 2 are randomly and iteratively assigned to replace remaining hydrogens and (b) for any given mutated peripheral constituent, dummy atoms 2 are randomly and systematically assigned to replace remaining hydrogens to generate a pool of fragment modules. In one example, fragment modules of the resulting library are indexed by chemical structure using, e.g., a simplified molecular input line entry system (SMILES).

In another aspect, at least one non-transitory computer-readable medium is provide that includes instructions that, when executed by at least one processor, are configured to cause the at least one processor to: (1) receive a dataset of pharmaceutically active parent molecules; (2) identify a template constituent from a respective parent molecule at a cleavage site for each of the parent molecules of the dataset; (3) fragment, according to a first set of rules, each of the identified constituents; store each of the fragmented constituents into a backbone library; (4) identify, according to a second set of rules, a set of backbone constituents and peripheral constituents; (5) store a copy set of peripheral constituents into a peripheral library such that the backbone library and peripheral library contain respective original and copy sets of the peripheral constituents; (6) mutate, according to a third set of rules, the stored constituents in each of the backbone and peripheral libraries; (7) systematically mark, according to a fourth set of rules, (a) each mutated constituent in the backbone library with a dummy atom 1 as a parent attachment and a dummy atom 2 as a peripheral enumeration and (b) each mutated constituent in the peripheral library with the dummy atom 2; (8) systematically combine respective constituents in the backbone and peripheral libraries at any respective dummy atom 2, to generate a dataset of fragment modules; and (9) store the dataset as a searchable fragment module library.

In one embodiment, the first set of rules for fragmenting each of the isolated constituents can be engendered in a computer-readable instruction that specifies cleaving all single bonds in any given constituent, wherein cleaving results in a larger fragment and a smaller fragment, and wherein the smaller fragment comprises at least one heavy atom and has a weight of <150 Da. The second set of rules can be engendered in a computer-readable instruction that specifies identifying each respective fragment as a backbone fragment unless (a) the number non-hydrogen atoms is ≤6 and (b) the cleavage site, which is the bond broken to create the fragment, is at a carbon atom, in which case the fragment is identified as a peripheral fragment.

The third set of rules can be engendered in a computer-readable instruction that specifies: (1), for any atoms of a given constituent, a mutation may occur only at a carbon; (2), for any aromatic carbon of a given constituent, (a) if the number of hydrogens is 0, then no mutation may occur; (b) if the number of hydrogens is 1, then (i) the carbon may be replaced by a nitrogen atom or a phosphorous atom or (ii) the hydrogen may be substituted with a halogen; (3), for any aliphatic ring carbon, (a) if the number of hydrogens is 0, then no mutation may occur; (b) if the number of hydrogens is one, then (i) the carbon may be replaced by a nitrogen atom or a phosphorous atom or (ii) the hydrogen may be substituted with a halogen; and (c) if the number of hydrogens is 2, then (i) the carbon may be replaced by a nitrogen atom or a phosphorous atom, (ii) the carbon and the two hydrogens may be replaced by an oxygen atom, a sulfur atom, or a selenium atom, (iii) the carbon and two hydrogens may be replaced by a carbonyl group; or (iv) one of the hydrogens may be substituted with a halogen, and (4), for any aliphatic chain carbon, (a) if the number of hydrogens is 0, then no mutation may occur; (b) if the number of hydrogens is one, then (i) the carbon may be replaced by a nitrogen atom or a phosphorous atom or (ii) the hydrogen may be substituted with a halogen; and (c) if the number of hydrogens is 2 or 3, then (i) the carbon may be replaced by a nitrogen atom or a phosphorous atom, (ii) the carbon and two hydrogens may be replaced by an oxygen atom, a sulfur atom, or a selenium atom, (iii) the carbon and two hydrogens may be replaced by a carbonyl group; or (iv) one of the hydrogens may be substituted with a halogen.

The fourth set of rules can be engendered in a computer-readable instruction that specifies: (1), for any given mutated constituent in the backbone library, dummy atoms 1 and dummy atoms 2 are randomly and iteratively assigned to replace remaining hydrogens, and (2), for any given mutated constituent in the peripheral library, dummy atoms 2 are randomly and systematically assigned to replace remaining hydrogens to generate a pool of fragment modules. In examples, the fragment molecules can be indexed by chemical structure using a simplified molecular input line entry system.

Further described is a fully automated approach to systematic lead optimization of active drug molecules using the fragment module libraries described herein. To that end, in another aspect, at least one non-transitory computer-readable medium is provide that includes instructions that, when executed by at least one processor, are configured to cause the at least one processor to: (1) receive and store structural data for a pharmaceutically active parent molecule; (2) identify a search constituent at a cleavage site of the pharmaceutically active parent molecule; (3) access a fragment module library to search structurally the library for complementary fragments to the search constituent; (4) output a datafile of fragment module search results; and (5) systematically combine each fragment module of the datafile with the parent molecule between the cleavage site and a respective dummy atom 1 of any given fragment module to generate a dataset of iterative parent derivative molecules. In one example, the dataset of iterative parent derivative molecules is output as a datafile and filtered to generate a subset of drug candidate molecules.

### BRIEF DESCRIPTION OF THE FIGURES

FIG. 1 illustrates a schematic diagram of a system environment implementing a cheminformatics subsystem.

FIG. 2 is a flow diagram of an example process for generating a fragment module library by a cheminformatics subsystem.

FIG. 3 is an illustration of an example computer-executable sorting operation of the disclosure performed on a dataset of fragmented template constituents of a pool of pharmaceutically active parent molecules into respective backbone libraries and peripheral libraries.

FIG. 4 is an illustration of an example computer-executable mutating operation of the disclosure performed on the datasets of sorted template constituents of FIG. 3.

FIG. 5 is an illustration of an example computer-executable marking operation of the disclosure performed on the datasets of mutated constituents of FIG. 4.

FIG. 6 presents the results of a fragment module library generated using an example software program of the disclosure (viz., ChemHopper™) versus Deepfrag.

FIG. 7 is an illustration of an example tranche-based library of indexed fragment modules of the disclosure.

FIG. 8 is an illustration of lead optimization and filtering operations of the disclosure.

FIG. 9 is a schematic illustration of Slougs framework for lead optimization utilizing ChemHopper™.

FIG. 10 shows a diagram of a computer device.

### DETAILED DESCRIPTION

The following discussion is presented to enable any person skilled in the art to make and use the technology disclosed and is provided in the context of a particular application and its requirements as well as a particular system environment and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown but is to be accorded the widest scope consistent with the principles and features disclosed herein.

Examples of programming languages, code and/or data libraries, and/or operating environments for use with the present technology include Python, Numpy, R, Java, Javascript, C#, C++, Julia, Shell, Go, TypeScript, and Scala.

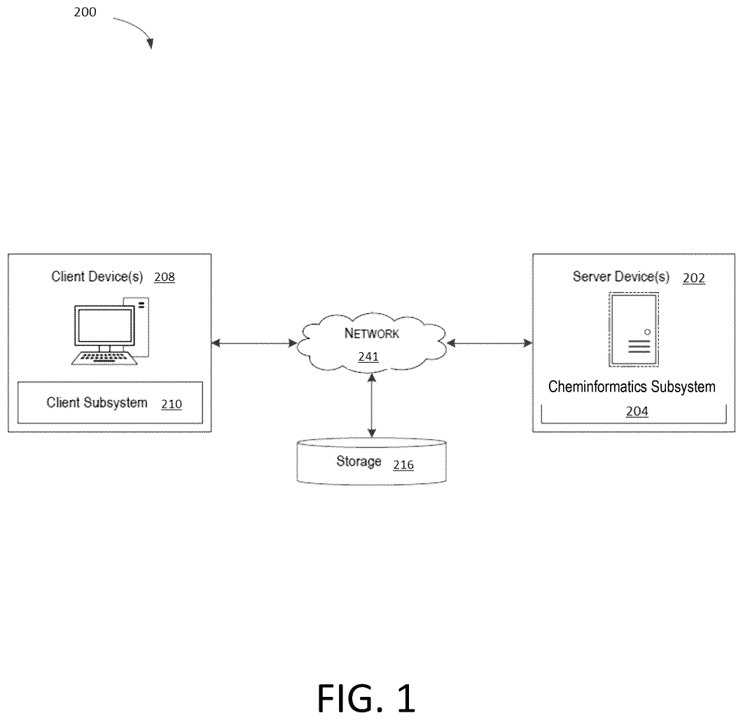

FIG. 1 illustrates a schematic diagram of a system environment (or “environment”) (a) generation of a fragment module library; (b) use of the fragment module library to lead optimize a drug of interest, and (c) filtering of optimized drug candidates. As illustrated, the environment 200 includes one or more server device(s) 202, which may be connected to one or more client (or user) device(s) 208 via a network 241 As shown in FIG. 1, the server device(s) 202 and the client device(s) 208 may communicate with each other via the network 241 or directly. The network 241 may comprise any suitable network over which computing devices may communicate. The network 241 may include a wired and/or wireless communication network. Example wireless communication networks may be comprised of one or more types of radio frequency (RF) communication signals using one or more wireless communication protocols, such as a cellular communication protocol, a wireless local area network (WLAN) or WIFI communication protocol, and/or another wireless communication protocol. In addition, or in the alternative to communicating across the network 241, the server device(s) 202, and the client device(s) 208 may bypass the network 241 and may communicate directly with one another.

As further illustrated in FIG. 1, the environment 200 may include storage 216. The storage 216 may store information for being accessed by the devices in the environment 200. The server device(s) 202 and the client device(s) 208 may communicate with the storage 216 (e.g., directly or via the network 241) to store and/or access information, including, e.g., any of the various libraries.

As further indicated by FIG. 1, the server device(s) 202 may generate, receive, analyze, store, and/or transmit digital data, such as datasets of parent drug molecules, parent derivative molecules, or other molecules of interest. The server device(s) 202 may communicate with the client device(s) 208. In particular, the server device(s) 202 may send data to the client device(s) 208, including, e.g., parent derivative molecules and filtered drug candidates, and the server device(s) 202 may receive input from users via client device(s) 208.

The server device(s) 202 may comprise a distributed collection of servers where the server device(s) 202 include a number of server devices distributed across the network 241 and located in the same or different physical locations. Further, the server device(s) 202 may comprise a content server, an application server, a communication server, a web-hosting server, or another type of server.

As further shown in FIG. 1, the server device(s) 202 may include a cheminformatics subsystem 204. The cheminformatics subsystem 204 may include software and/or hardware utilized by the server device(s) 202 for processing datasets of pharmaceutically active parent molecules; identifying template constituents from a respective parent molecule at a cleavage site for each of the parent molecules of the datasets; fragmenting the identified constituents; sorting fragmented constituents into backbone libraries and peripheral libraries; mutating sorted backbone constituents and peripheral constituents; systematically marking mutated backbone constituents and peripheral constituents with dummy atoms; systematically combining marked backbone constituents and peripheral constituents; and/or generation and output of fragment modules datasets.

The cheminformatics subsystem 204 may also include software and/or hardware utilized by the server device(s) 202 for receiving structural data for pharmaceutically active parent molecules for lead optimization; identification of search constituents at a cleavage site of the pharmaceutically active parent molecules; accessing fragment module libraries disclosed herein to search structurally the library for complementary fragments to the search constituent; outputting datafiles of fragment module search results; systematically combining fragment modules of the datafile with parent molecules between the cleavage site and a marked dummy atom of any given fragment module; generation of datasets of iterative parent derivative molecules; filtering of parent derivative molecule datasets to generate subsets of drug candidate molecules; and outputting datafiles of drug candidate molecules.

The cheminformatics subsystem 204 may be included in a single server device 202 or may be distributed across multiple devices. The cheminformatics subsystem 204 may span multiple layers of software and/or hardware for servicing requests at the cheminformatics subsystem 204. The cheminformatics subsystem 204 may support drug optimization and filtering services for clients or other users.

Each client device 208 may generate, store, receive, and/or send digital data. For example, the client device 208 may communicate with the server device(s) 202 to receive one or more datafiles comprising output from the cheminformatics subsystem 204, including datafiles comprising datasets of parent derivative molecules and/or drug candidate molecules. Furthermore, the client device 208 may present or display information pertaining to parent derivative molecules and/or drug candidate molecules within a graphical user interface to a human user associated with the client device 208. The client device(s) 208 may comprise various types of client devices. In examples, the client device 208 may include non-mobile devices, such as desktop computers or servers, or other types of client devices. In other examples, the client device 208 may include mobile devices, such as laptops, tablets, mobile telephones, or smartphones.

As further illustrated in FIG. 1, each client device 208 may include a client subsystem 210. The client subsystem 210 may include software and/or hardware utilized by the client device 208 for processing datasets of parent derivative molecules and/or drug candidate molecules. The client subsystem 210 may span multiple layers of software and/or hardware. The client subsystem 210 may be included in a single client device 208 or may be distributed across multiple client devices 208.

Client processes may be operated on one or more of the client device 208 and/or the server device 202 for requesting drug optimization and filtering services from the cheminformatics subsystem 204. For example, client processes executing on any of the client device(s) 208 and/or server device(s) 202 may transmit requests for performing drug optimization and/or filtering services for particular biologically active molecules of interest. The cheminformatics subsystem 204 may load and/or execute different bitstreams to perform various types of analysis to support requests from the client processes.

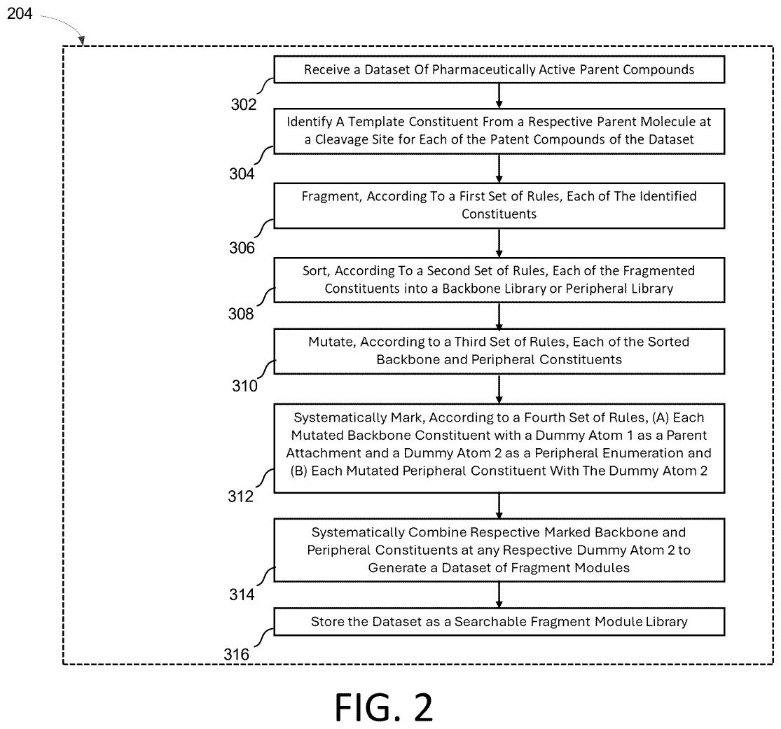

Referring to FIG. 2, in one aspect, a cheminformatic subsystem 204 may include one or more engines for implementing one or more methods or procedures. For example, the bioinformatics subsystem 204 may include a receiving engine 302, an identification engine 304, a fragmenting engine 306, a sorting engine 308, a mutating engine 310, a marking engine 312, a combining engine 314, and a storing engine 316 for implementing one or more methods or procedures.

In examples, the cheminformatic subsystem 204 may cause receiving engine 302 to receive a dataset of pharmaceutically active parent molecules, e.g., FDA approved drug molecules. The cheminformatic subsystem 204 may then cause the identifying engine 304 to identify a template constituent from a respective parent molecule at a cleavage site for each of the parent molecules of the dataset.

Next, the cheminformatic subsystem 204 may cause the fragmenting engine 306 to fragment each of the identified constituents. The fragmenting engine 306 may operate according to an executable instruction or set of instructions. In one example, an instruction implements a first set of rules for fragmenting each of the isolated constituents, which may specify cleavage all single bonds in any given constituent that result in fragmenting the constituent into a larger fragment and a smaller fragment, wherein the smaller fragment (a) contains at least one heavy atom and (b) has a weight of <150 Da.

Next, the cheminformatic subsystem 204 may cause the sorting engine 308 to sort each of the fragmented constituents into a backbone library or a peripheral library. The sorting engine 308 may operate according to an executable instruction or set of instructions. In one example, an instruction implements a second set of rules for sorting each of the fragmented constituents respective backbone libraries and peripheral libraries, which may specify that a given fragment be identified as a backbone fragment unless (a) if the number non-hydrogen atoms is ≤6 and (b) the cleavage site, i.e., the bond broken to create the fragment, is at a carbon atom, in which case the fragment is identified as a peripheral fragment. In one example, all fragmented constituents may be stored to the backbone library and a copy set of peripheral fragments may be saved, according to the second set of rules, into the peripheral library such that both the backbone library and the peripheral library contain a respective original and copy set of peripheral fragments.

Next, the cheminformatic subsystem 204 may cause the mutating engine 310 to mutate each of the sorted backbone constituents and peripheral constituents. The mutating engine 310 may operate according to an executable instruction or set of instructions. In one example, an instruction implements a third set of rules for mutating each of the sorted backbone constituents and peripheral constituents which may specify that (1) for any atoms of a given constituent, a mutation may occur only at a carbon; (2), for any aromatic carbon of a given constituent, (a) if the number of hydrogens is 0, then no mutation may occur; (b) if the number of hydrogens is 1, then (i) the carbon may be replaced by a nitrogen atom or (ii) the hydrogen may be substituted with a halogen; (3) for any aliphatic ring carbon, (a) if the number of hydrogens is 0, then no mutation may occur; (b) if the number of hydrogens is one, then (i) the carbon may be replaced by a nitrogen atom or (ii) the hydrogen may be substituted with a halogen; and (c) if the number of hydrogens is 2, then (i) the carbon may be replaced by a nitrogen atom, (ii) the carbon and the two hydrogens may be replaced by an oxygen atom, (iii) the carbon and two hydrogens may be replaced by a carbonyl group; or (iv) one of the hydrogens may be substituted with a halogen and (4) for any aliphatic chain carbon, (a) if the number of hydrogens is 0, then no mutation may occur; (b) if the number of hydrogens is one, then (i) the carbon may be replaced by a nitrogen atom or (ii) the hydrogen may be substituted with a halogen; and (c) if the number of hydrogens is 2 or 3, then (i) the carbon may be replaced by a nitrogen atom, (ii) the carbon and two hydrogens may be replaced by an oxygen atom, (iii) the carbon and two hydrogens may be replaced by a carbonyl group; or (iv) one of the hydrogens may be substituted with a halogen. In examples, all possible mutated permutations may be generated in accordance with the instruction. In addition to nitrogen, oxygen, and phosphorous mutations illustrated, other non-reactive metals may be used to form mutations as appropriate, e.g., sulfur, phosphorous, selenium.

Next, the cheminformatic subsystem 204 may cause the marking engine 312 to systematically mark (a) each mutated backbone constituent with a dummy atom 1 as a parent attachment and a dummy atom 2 as a peripheral enumeration and (b) each mutated peripheral constituent with the dummy atom 2. The marking engine 312 may operate according to an executable instruction or set of instructions. In one example, an instruction implements a fourth set of rules for mutating each of the sorted backbone constituents and peripheral constituents which may specify that (a) for any given mutated backbone constituent, dummy atoms 1 and 2 are randomly and iteratively assigned to replace remaining hydrogens and (b) for any given mutated peripheral constituent, dummy atoms 2 are randomly and systematically assigned to replace remaining hydrogens to generate a pool of fragment modules.

Next, the cheminformatic subsystem 204 may cause the combining engine 314 systematically combine respective marked backbone constituents and peripheral constituents at any respective dummy atom 2, to generate a dataset of fragment modules. The cheminformatic subsystem 204 then may cause the storing engine 316 to store the dataset as a searchable fragment module library. In one example, fragment modules of the resulting library are indexed by chemical structure using, e.g., a simplified molecular input line entry system (SMILES).

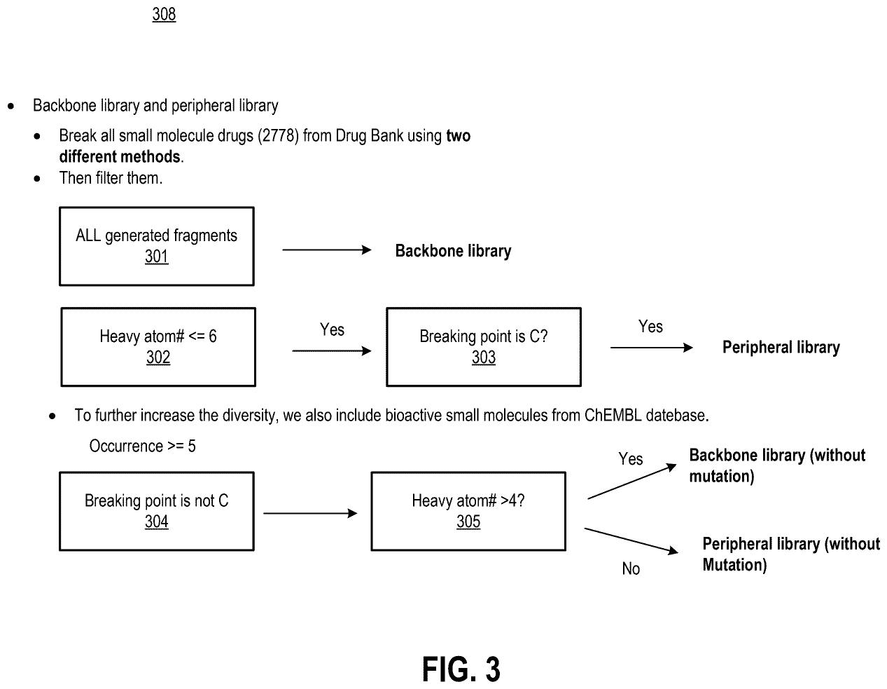

FIG. 3 provides an illustrated process performed by sorting engine 308. As shown, the essential backbone libraries and peripheral libraries are generated using the relatively small number FDA approved small molecule drugs (˜3,000 unique molecules) from Drugbank. Once obtained, the molecules may be broken up into smaller fragments. In the example, referring back to FIG. 2, an instruction, as executed by fragmentation engine 306, implements a set of rules specifying cleavage all single bonds in any given constituent that result in fragmenting the constituent into a larger fragment and a smaller fragment, wherein the smaller fragment (a) contains at least one heavy atom and (b) has a weight of <150 Da. Other molecular fragmentation techniques for use consistent with the present disclosure include. e.g., FCS, CS, BPE, SPE, MMPs, RECAP, BRICS, eMolFrag, BP_NLM, MacFrag, FG splitting, FASMIFRA, CReM, UNIFAC, and VOLT.

With reference to FIG. 3, those fragments 301 may then be characterized into either backbone fragments or peripheral fragments by sorting engine 308, utilizing two rules: (1) at 302, if the number of non-hydrogen atoms contained in the fragment is less than or equal to 6, and, at 303, the cleavage site, the bond broken to create the two fragments, contains a carbon atom, then the fragment is a peripheral fragment; (2) at 304, if the breaking point is not a carbon atom and, at 305, the number of heavy atoms is greater than four, the fragment is a backbone fragment, whereas, at 305, if the number of heavy atoms is less than or equal to four, the fragment is a peripheral fragment. In the example of FIG. 3, all fragmented constituents may be stored to the backbone library and a copy set of peripheral fragments may be saved, according to the second set of rules, into the peripheral library such that both the backbone library and the peripheral library contain a respective original and copy set of peripheral fragments.

These libraries may be further augmented using common bioactive structures contained, e.g., in the ChEMBL database. As illustrated in FIG. 3, in one example, fragments generated from structures in the ChEMBL database may be first filtered based upon frequency, with all fragments with fewer than five unique occurrences removed. After filtering, fragments are characterized based upon slight variations of the previous rules: if the cleavage site does not contain a carbon atom and the number of non-hydrogen atoms is greater than or equal to 4, then the element is a backbone fragment, otherwise it is a peripheral fragment.

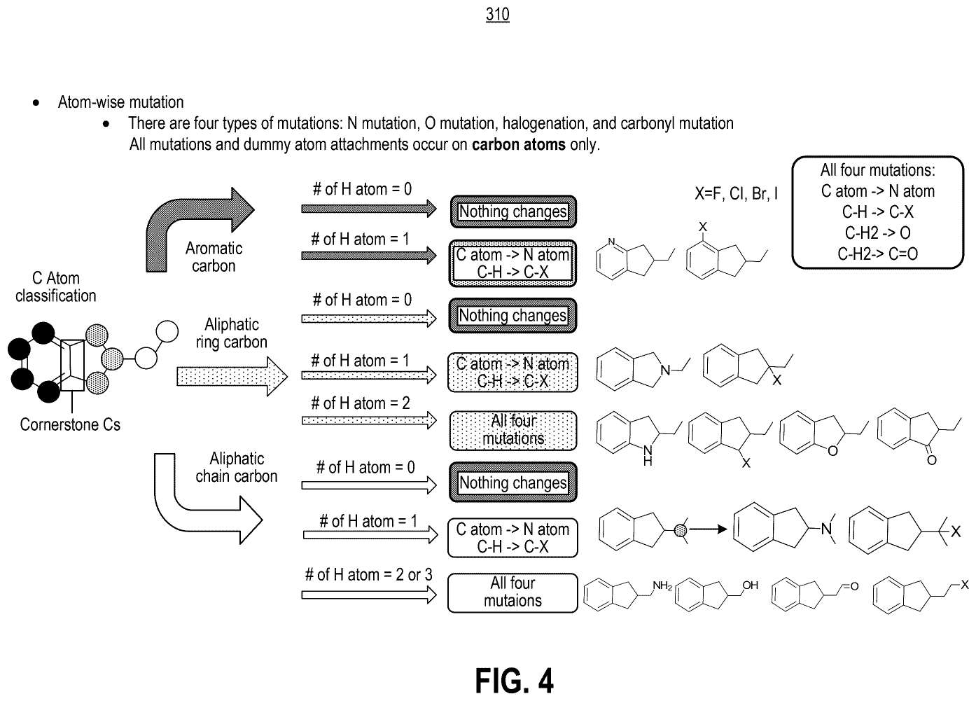

FIG. 4 provides an illustrated process performed by mutating engine 310. As illustrated, after creation of the backbone libraries and peripheral libraries, the depth of the libraries is significantly magnified through simulated chemical mutation performed by mutating engine 310. The mutation process may involve scanning a given fragment, classifying each carbon into one of four unique classes: cornerstone carbons, aromatic carbons, ring carbons, and peripheral carbons. Cornerstone carbons are those carbons where all four standard bonds are involved in the structure of the fragment and thus cannot be altered without foundationally changing the fragment.

Aromatic carbons are carbons that are contained in a closed ring that exhibits aromatic properties, i.e., fulfilling Huckel's rules: planar structure, each atom in the ring is conjugated, multiple resonance structures, and (4n+2) 7E electrons. Aromatic carbons typically have one carbon-hydrogen bond and may be mutated through a variety of processes including, but not limited to, halogenation (substitution of a hydrogen atom with a halogen atom such as fluorine or chlorine), or carbon-nitrogen substitution, as long as it maintains the aromatic nature of the ring. Ring carbons are carbon atoms in the fragment that are part of a ring that isn't aromatic, i.e., violates Huckel's rules.

Ring carbons typically have 0, 1, or 2 carbon-hydrogen bonds and may be mutated through a variety of process including, but not limited to, carbon-nitrogen substitution, carbon-oxygen substitution, or carbonyl substitution (exchanging two hydrogen atoms bound to a single carbon with an oxygen with a double bond to the carbon).

Finally, peripheral carbons are carbon atoms that are not part of a ring and may have 0, 1 or 2 carbon-hydrogen bonds. Peripheral carbons may be mutated through a variety of process including but not limited to carbon-nitrogen substitution, carbon-oxygen substitution, and halogenation. Other reactive nonmetals may also be utilized for mutation, including, but not limited to, sulfur, phosphorous, and selenium.

In one example embodiment, at least one non-transitory computer-readable medium is provide that includes instructions that, when executed by at least one processor, are configured to cause the at least one processor to: (1) receive a dataset of pharmaceutically active parent molecules; (2) identify a template constituent from a respective parent molecule at a cleavage site for each of the parent molecules of the dataset; (3) fragment, according to a first set of rules, each of the identified constituents; store each of the fragmented constituents into a backbone library; (4) identify, according to a second set of rules, a set of backbone constituents and peripheral constituents; (5) store a copy set of peripheral constituents into a peripheral library such that the backbone library and peripheral library contain respective original and copy sets of the peripheral constituents; (6) mutate, according to a third set of rules, the stored constituents in each of the backbone and peripheral libraries; (7) systematically mark, according to a fourth set of rules, (a) each mutated constituent in the backbone library with a dummy atom 1 as a parent attachment and a dummy atom 2 as a peripheral enumeration and (b) each mutated constituent in the peripheral library with the dummy atom 2; (8) systematically combine respective constituents in the backbone and peripheral libraries at any respective dummy atom 2, to generate a dataset of fragment modules; and (9) store the dataset as a searchable fragment module library.

The first set of rules for fragmenting each of the isolated constituents may be engendered in a computer-readable instruction that specifies cleaving all single bonds in any given constituent, wherein cleaving results in a larger fragment and a smaller fragment, and wherein the smaller fragment comprises at least one heavy atom and has a weight of <150 Da. The second set of rules may be engendered in a computer-readable instruction that specifies identifying each respective fragment as a backbone fragment unless (a) the number non-hydrogen atoms is ≤6 and (b) the cleavage site, which is the bond broken to create the fragment, is at a carbon atom, in which case the fragment is identified as a peripheral fragment.

The third set of rules may be engendered in a computer-readable instruction that specifies: (1), for any atoms of a given constituent, a mutation may occur only at a carbon; (2), for any aromatic carbon of a given constituent, (a) if the number of hydrogens is 0, then no mutation may occur; (b) if the number of hydrogens is 1, then (i) the carbon may be replaced by a nitrogen atom or a phosphorous atom or (ii) the hydrogen may be substituted with a halogen; (3), for any aliphatic ring carbon, (a) if the number of hydrogens is 0, then no mutation may occur; (b) if the number of hydrogens is one, then (i) the carbon may be replaced by a nitrogen atom or a phosphorous atom or (ii) the hydrogen may be substituted with a halogen; and (c) if the number of hydrogens is 2, then (i) the carbon may be replaced by a nitrogen atom or a phosphorous atom, (ii) the carbon and the two hydrogens may be replaced by an oxygen atom, a sulfur atom, or a selenium atom, (iii) the carbon and two hydrogens may be replaced by a carbonyl group; or (iv) one of the hydrogens may be substituted with a halogen, and (4), for any aliphatic chain carbon, (a) if the number of hydrogens is 0, then no mutation may occur; (b) if the number of hydrogens is one, then (i) the carbon may be replaced by a nitrogen atom or a phosphorous atom or (ii) the hydrogen may be substituted with a halogen; and (c) if the number of hydrogens is 2 or 3, then (i) the carbon may be replaced by a nitrogen atom or a phosphorous atom, (ii) the carbon and two hydrogens may be replaced by an oxygen atom, a sulfur atom, or a selenium atom, (iii) the carbon and two hydrogens may be replaced by a carbonyl group; or (iv) one of the hydrogens may be substituted with a halogen.

The fourth set of rules may be engendered in a computer-readable instruction that specifies: (1), for any given mutated constituent in the backbone library, dummy atoms 1 and dummy atoms 2 are randomly and iteratively assigned to replace remaining hydrogens, and (2), for any given mutated constituent in the peripheral library, dummy atoms 2 are randomly and systematically assigned to replace remaining hydrogens to generate a pool of fragment modules. In examples, the fragment molecules may be indexed by chemical structure using a simplified molecular input line entry system.

In one example, using the in silico chemical mutation process herein, a 2-ethylindan fragment may be magnified to 25 or more structures: 6-ethyl-6,7-dihydro-5H-cyclopenta[b]pyridine; 2-ethyl-2,3-dihydro-4-fluoro-1H-indene; 2-ethyl-2,3-dihydro-4-chloro-1H-indene N-ethyl-isoindoline; 2-ethyl-2,3-dihydro-4-bromo-1H-indene; 2-ethyl-2,3-dihydro-4-iodo-1H-indene; 2-fluoro-2,3-dihydro-1H-indene; 2-chloro-2,3-dihydro-1H-indene; 2-bromo-2,3-dihydro-1H-indene; 2-iodo-2,3-dihydro-1H-indene; -ethyl-2,3,-dihydro-1H-indole; 1-fluoro-2-ethyl-2,3-dihydro-1H-indene; 1-chloro-2-ethyl-2,3-dihydro-1H-indene; 1-bromo-2-ethyl-2,3-dihydro-1H-indene; 1-iodo-2-ethyl-2,3-dihydro-1H-indene; 2-ethyl-2,3-dihydrobenzofuran; 9-ethyl-1-indanone; 2-i-propyl-2,3,-dihydro-1H-indene; N,N-dimethyl-2-aminoindan; 2-[2-(2,3-dihydro-1H-indenyl)]-2-fluoro-propane; 2-[2-(2,3-dihydro-1H-indenyl)]-2-chloro-propane; 2-[2-(2,3-dihydro-1H-indenyl)]-2-bromo-propane; 2-[2-(2,3-dihydro-1H-indenyl)]-2-iodo-propane; [2-(2,3-dihydro-1H-indenyl)]methylamine; [2-(2,3-dihydro-1H-indenyl)]methanol; [2-(2,3-dihydro-1H-indenyl)]ethanal; 1-[2-(2,3-dihydro-1H-indenyl)]-2-fluoro-ethane; 1-[2-(2,3-dihydro-1H-indenyl)]-2-chloro-ethane; 1-[2-(2,3-dihydro-1H-indenyl)]-2-bromo-ethane; and 1-[2-(2,3-dihydro-1H-indenyl)]-2-iodo-ethane.

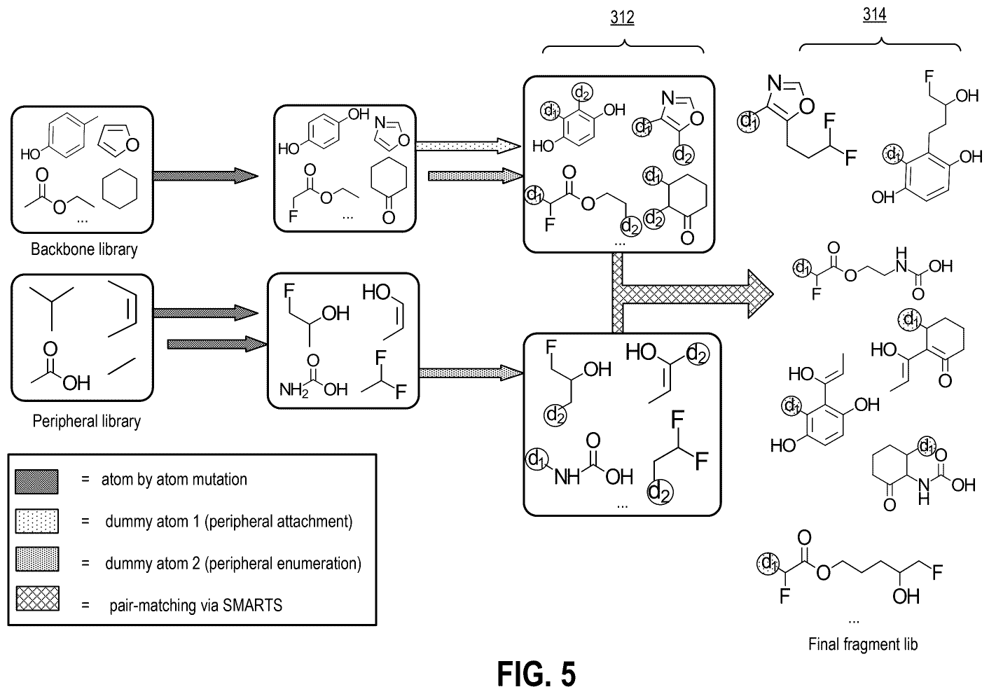

FIG. 5 provides an illustrated process performed by marking and combining engines 312 and 314. Here, having significantly increased the molecule diversity of both backbone and peripheral libraries with the mutation process, the number of module fragments is significantly increased again through the iterative combination of backbone fragments and peripheral fragments. To facilitate this, a pair of randomly selected hydrogen atoms in each backbone fragment may be removed and replaced with dummy atom 1 and dummy atom 2, respectively. Dummy atom 1 serves as a future connection point to a parent molecule in the final derivative library, while dummy atom 2 serves a connection point to a peripheral fragment. Simultaneously, a randomly selected hydrogen atom in each peripheral fragment is removed and replaced with dummy atom 2. Once all possible arrangements of dummy atoms are placed for both backbone and peripheral fragments, fragments are then combined at the corresponding dummy atom 2 locations. This approach creates a massive database of potential modules with a specified attachment point, dummy atom 1, that may be attached to any parent molecule of interest to create a mega library of fragment modules, with similar structure to the parent molecule.

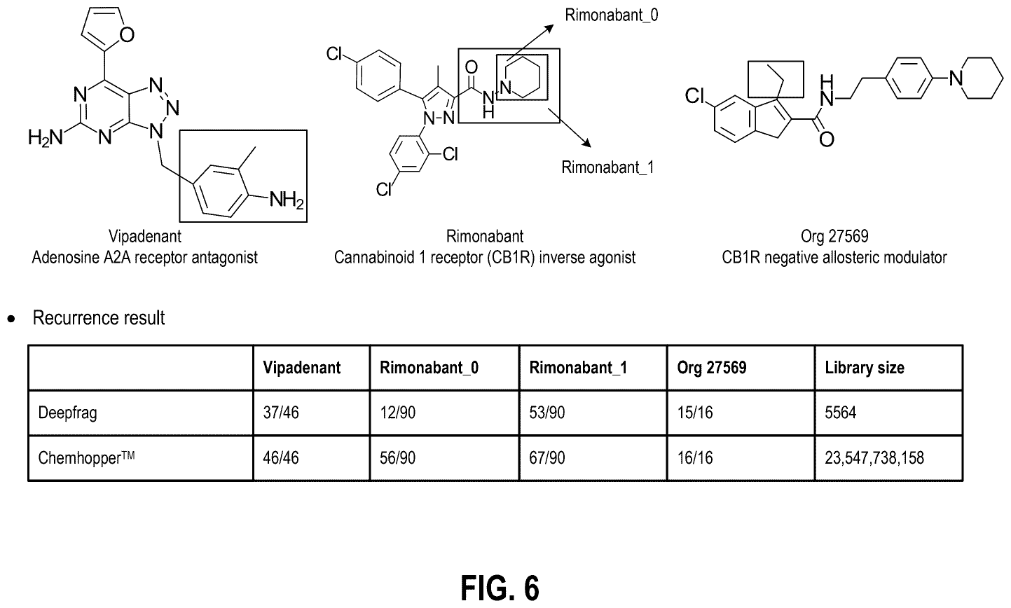

The cheminformatics subsystem 204 may include software technology utilized by the server device(s) 202 for performing the processes described herein, an example of which is ChemHopper™. Unlike other software products, ChemHopper™ is not based on artificial intelligence or machine learning but is rather a systems and knowledge driven approach to identify potential lead molecules. As a result, ChemHopper™ cannot be evaluated based upon its ability to predict if a particular molecule will be a good lead candidate, rather it should be evaluated on its ability to generate known lead candidates. With reference to FIG. 6, and Table 1, below, running ChemHopper™ on three known small molecules, (Vipadenant, an Adenosine A2A receptor agonist; Rimonabant, a cannabinoid 1 receptor (CB1R) inverse agonist; and Org 27569, a CB1R negative allosteric modulator) with a variety of known lead derivates, showed significantly higher recurrence of those derivates than those predicted by Deepfrag software.

TABLE 1

| | Vipadenant | Rimonabant_0 | Rimonabant_1 | Org 27569 | Library Size |
|---|---|---|---|---|---|
| Deepfrag | 37/46 | 12/90 | 53/90 | 15/16 | 5564 |
| Chemhopper ™ | 46/46 | 56/90 | 67/90 | 16/16 | 23, 547, 738, 158 |

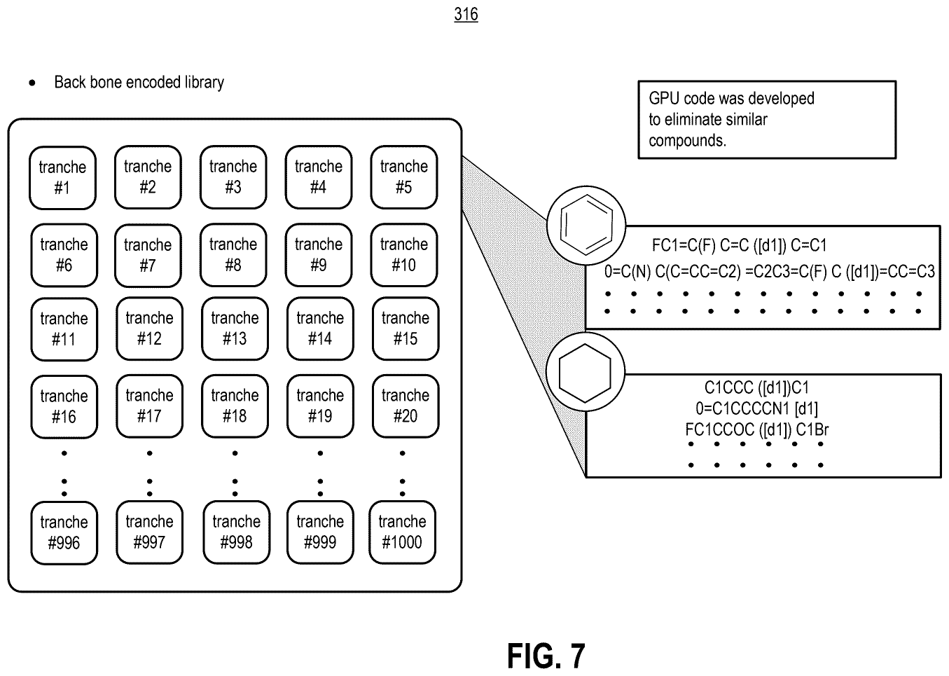

FIG. 7 provides an illustrated process performed by sorting engine 316. As illustrated, the mega-library of fragmented modules generated via combining engine 314 may be sorted by tranche, where each tranche is populated with modules sharing the same back bone structure. In one example, sorting engine 316 may execute a dedupe process, eliminating from each tranche duplicate or substantially duplicative modules. Each module may be also encoded with an input line entry for molecular structure (e.g., SMILES) for purposes of performing a search function.

In certain embodiments a machine learning model may be trained to directly generate fragments to replace parts of molecules of interest, conditioned on the 3D structure of the binding pocket and the molecule fragment of interest. Simultaneously, this model may be capable of screening a given fragment database.

According to an example, a proposed training set may include or comprise the CrossDocked2020 dataset. The 3D ligand-protein complexes from this dataset may be used to train a conditional variational autoencoder (cVAE), with the SMILES strings of the fragments (broken from the parent molecules) as input. Because the cVAE provides latent vectors of the fragments, which also encode 3D information of the protein and ligand, latent vectors may be leveraged to screen known fragment libraries. Novel fragments may then be generated by randomly sampling from a unit Gaussian distribution in the latent space and decoding the vector.

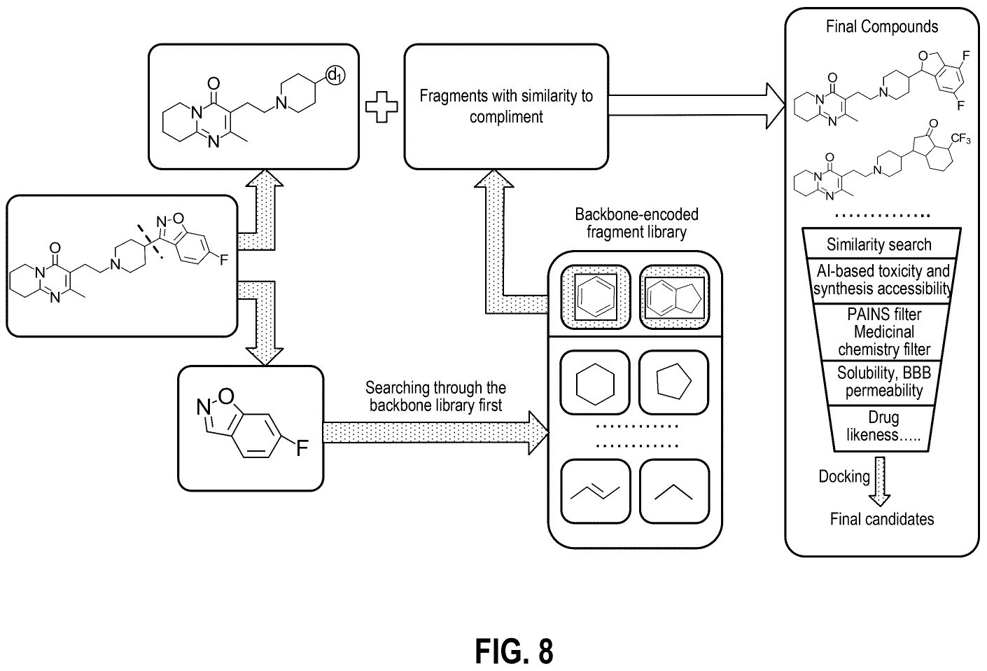

FIG. 8 is an illustration of drug optimization and filtering services for clients or other users supported by the cheminformatics subsystem 204. In one example, an active parent molecule is received, e.g., from a client device 208 (of FIG. 1), and a search constituent at a cleavage site of the pharmaceutically active parent molecule is first identified. Next, a fragment module library may be accessed to search structurally the library for complementary fragments to the search constituent. The fragment modules identified in the search are then systematically combined with the parent molecule between the cleavage site (marked in the example with a dummy atom 1) and a respective dummy atom 1 of any given fragment module to generate a dataset of iterative parent derivative molecules. The dataset may then be written to a datafile made accessible to the client device 208 or for further downstream processing. In the example of FIG. 8, the datafile may be further processed in silico through one or more filters to derive a subset of drug candidate molecules.

Any number of filters may be used consistent with the present disclosure. In the example of FIG. 8, the dataset of iterative parent derivative molecules is first filtered through a similarity search. Next, molecules passing filter are filtered for predictive toxicity (e.g., LimTox, pkCSM, admetSAR, and Toxtree) and synthesis accessibility using one or more AI-based programs. Molecules passing the filters are then run serially through a PAINS filter, Medicine chemistry filter, solubility and BBB permeability filter, and drug likeness filter. Molecules passing the filters are written to a datafile as final drug candidate molecules made accessible to the client device 208.

An example of AI is intelligence as manifested by computer systems. The AI-driven signal enhancement is implemented at least in part, for example, via one or more computer systems implementing and/or based on one or more machine learning techniques such as deep learning using one or more Neural Networks (NNs). Various examples of NNs include Convolutional Neural Networks (CNNs) generally (e.g., any NN having one or more layers performing convolution), as well as NNs having elements that include one or more CNNs and/or CNN-related elements (e.g., one or more convolutional layers), such as various implementations of Generative Adversarial Networks (GANs) generally, as well as various implementations of Conditional Generative Adversarial Networks (CGANs), cycle-consistent Generative Adversarial Networks (CycleGANs), and autoencoders. In various scenarios, any NN having at least one convolutional layer is referred to as a CNN. The various examples of NNs further include transformer-based NNs generally (e.g., any NN having one or more layers performing an attention operation such as a self-attention operation or any other type of attention operation), as well as NNs having elements that include one or more transformers and/or transformer-related elements. The various examples of NNs further include Recurrent Neural Networks (RNNs) generally (e.g., any NN in which output from a previous step is provided as input to a current step and/or having hidden state), as well as NNs having one or more elements related to recurrence. The various examples of NNs further include graph neural networks and diffusion neural networks. The various examples of NNs further include MultiLayer Perceptron (MLP) neural networks. In some implementations, a GAN is implemented at least in part via one or more MLP elements.

Examples of elements of NNs include layers, such as processing, activation, and pooling layers, as well as loss functions and objective functions. According to implementation, functionality corresponding to one or more processing layers, one or more pooling layers, and/or one or more an activation layers is included in layers of a NN. According to implementation, some layers are organized as a processing layer followed by an activation layer and optionally the activation layer is followed by a pooling layer. For example, a processing layer produces layer results via an activation function followed by pooling. Additional example elements of NNs include batch normalization layers, regularization layers, and layers that implement dropout, as well as recurrent connections, residual connections, highway connections, peephole connections, and skip connections. Further additional example elements of NNs include gates and gated memory units, such as Long Short-Term Memory (LSTM) blocks or Gated Recurrent Unit (GRU) blocks, as well as residual and/or attention blocks.

Examples of processing layers include convolutional layers generally, upsampling layers, downsampling layers, averaging layers, and padding layers. Examples of convolutional layers include 1D convolutional layers, 2D convolutional layers, 3D convolutional layers, 4D convolutional layers, 5D convolutional layers, multi-dimensional convolutional layers, single channel convolutional layers, multi-channel convolutional layers, 1×1 convolutional layers, atrous convolutional layers, dilated convolutional layers, transpose convolutional layers, depthwise separable convolutional layers, pointwise convolutional layers, 1×1 convolutional layers, group convolutional layers, flattened convolutional layers, spatial convolutional layers, spatially separable convolutional layers, cross-channel convolutional layers, shuffled grouped convolutional layers, and pointwise grouped convolutional layers. Convolutional layers vary according to various convolutional layer parameters, for example, kernel size (e.g., field of view of the convolution), stride (e.g., step size of the kernel when traversing an image), padding (e.g., how sample borders are processed), and input and output channels. An example kernel size is 3×3 pixels for a 2D image. An example default stride is one. In various implementations, strides of one or more convolutional layers are larger than unity (e.g., two). A stride larger than unity is usable, for example, to reduce sizes of non-channel dimensions and/or downsampling. A first example of padding (sometimes referred to as ‘padded’) pads zero values around input boundaries of a convolution so that spatial input and output sizes are equal (e.g., a 5×5 2D input image is processed to a 5×5 2D output image). A second example of padding (sometimes referred to as ‘unpadded’) includes no padding in a convolution so that spatial output size is smaller than input size (e.g., a 6×6 2D input image is processed to a 4×4 2D output image).

Example activation layers implement, e.g., non-linear functions, such as a rectifying linear unit function (sometimes referred to as ReLU), a leaky rectifying linear unit function (sometimes referred to as a leaky-ReLU), a parametric rectified linear unit (sometimes referred to as a PreLU), a Gaussian Error Linear Unit (GELU) function, a sigmoid linear unit function, a sigmoid shrinkage function, an SiL function, a Swish-1 function, a Mish function, a Gaussian function, a softplus function, a maxout function, an Exponential Linear Unit (ELU) function, a Scaled Exponential Linear Unit (SELU) function, a logistic function, a sigmoid function, a soft step function, a softmax function, a Tangens hyperbolicus function, a tanh function, an arctan function, an ElliotSig/Softsign function, an Inverse Square Root Unit (ISRU) function, an Inverse Square Root Linear Unit (ISRLU) function, and a Square Nonlinearity (SQNL) function.

Examples of pooling layers include maximum pooling layers, minimum pooling layers, average pooling layers, and adaptive pooling layers.

Examples of loss functions include loss functions in accordance with one or more loss terms, such as a logistic regression/log loss, a multi-class cross-entropy/softmax loss, a binary cross-entropy loss, a mean squared error loss, a mean absolute error loss, a mean absolute percentage error loss, a mean squared logarithmic error loss, an L1 loss, an L2 loss, a smooth L1 loss, a Huber loss, a patch-based loss, a pixel-based loss, a pixel-wise loss, a perceptual loss, a Wasserstein loss (sometimes termed an Earth Mover distance loss), and a fiducial-based loss. Some loss functions are based on comparing intermediary activations of a NN, such as between layers. Some loss functions are based on a spatial domain. Some loss functions are based on a frequency domain.

Example objective functions include maximizing a likelihood, maximizing a log likelihood, maximizing a probability, maximizing a log probability, and minimizing one or error terms (e.g., as determined via one or more loss functions). Further example objective functions include an Evidence Lower Bound Objective (ELBO) function and any objective function based on a Kullback-Leibler (KL) divergence term. According to implementation, a penalty is applied to an objective function. Example penalties applicable to various objective functions include a ridge regression penalty and a lasso regression penalty.

Example techniques to train NNs, such as to determine and/or update parameters of the NNs, include backpropagation-based gradient update and/or gradient descent techniques, such as Stochastic Gradient Descent (SGD), synchronous SGD, asynchronous SGD, batch gradient descent, and mini-batch gradient descent. The backpropagation-based gradient techniques are usable alone or in any combination, e.g., stochastic gradient descent is usable in a mini-batch context. Example optimization techniques usable with, e.g., backpropagation-based gradient techniques (such as gradient update and/or gradient descent techniques) include Momentum, Nesterov accelerated gradient, Adagrad, Adadelta, RMSprop, Adam, AdaMax, Nadam, and AMSGrad.

According to implementation, elements of NNs, such as layers, loss functions, and/or objective functions, variously correspond to one or more hardware elements, one or more software elements, and/or various combinations of hardware elements and software elements. For a first example, a convolution layer, such as a N×M×D convolutional layer, is implemented as hardware logic circuitry comprised in an Application Specific Integrated Circuit (ASIC). For a second example, a plurality of convolutional, activation, and pooling layers are implemented in a TensorFlow machine learning framework on a collection of Internet-connected servers. For a third example, a first one or more portions a NN, such as one or more convolution layers, are respectively implemented in hardware logic circuitry according to the first example, and a second one or more portions of the NN, such as one or more convolutional, activation, and pooling layers, are implemented on a collection of Internet-connected servers according to the second example. Various implementations are contemplated that use various combinations of hardware and software elements to provide corresponding price and performance points.

Example characterizations of a NN architecture include any one or more of topology, interconnection, number, arrangement, dimensionality, size, value, dimensions and/or number of hyperparameters, and dimensions and/or number of parameters of and/or relating to various elements of a NN (e.g., any one or more of layers, loss functions, and/or objective functions of the NN).

Example implementations of a NN architecture include various collections of software and/or hardware elements that collectively perform operations according to the NN architecture. Various NN implementations vary according to machine learning framework, programming language, runtime system, operating system, and underlying hardware resources. The underlying hardware resources variously include one or more computer systems, such as having any combination of Central Processing Units (CPUs), Graphics Processing Units (GPUs), Field Programmable Gate Arrays (FPGAs), Coarse-Grained Reconfigurable Architectures (CGRAs), Application-Specific Integrated Circuits (ASICs), Application Specific Instruction-set Processors (ASIPs), and Digital Signal Processors (DSPs), as well as computing systems generally, e.g., elements enabled to execute programmed instructions specified via programming languages. Various NN implementations are enabled to store programming information (such as code and data) on non-transitory computer readable media and are further enabled to execute the code and reference the data according to programs that implement NN architectures.

Examples of machine learning frameworks, platforms, runtime environments, and/or libraries, such as enabling investigation, development, implementation, and/or deployment of NNs and/or NN-related elements, include TensorFlow, Theano, Torch, PyTorch, Keras, MLpack, MATLAB, IBM Watson Studio, Google Cloud AI Platform, Amazon SageMaker, Google Cloud AutoML, RapidMiner, Azure Machine Learning Studio, Jupyter Notebook, and Oracle Machine Learning.

To effectively screen the mega library generated by ChemHopper™ and identify fragments with potential binding affinity for protein targets, Slougs may be incorporated (Structure-directed Lead Optimization algorithm Unifying fragment Generation and Screening). The Slougs framework utilizes a transformer-based variational autoencoder (VAE), and an E(3)-equivalent graph neural network (GNN) to capture the 3D structural context of the binding pocket and the parent fragment, and to predict the latent vector. This latent vector is used to screen a predefined fragment library—encoded by the same VAE encoder—by computing cosine similarity for fragment selection.

When applying Slougs to screen the library generated by ChemHopper™, the dummy atom marking the connection point of the fragment library (i.e., Ag) is replaced by the symbol “*”, which denotes a dummy atom (atom number 0) in the RDkit library. A 3D protein structure file (.pdb) containing all residues within 6 Å of the ligand of interest is used as the binding pocket input. In parallel, an .sdf file containing the 3D parent fragment—with the connection point linked to a dummy atom in 3D space—is provided as the parent ligand input. The latent vector predicted by Slougs from the binding pocket and parent ligand inputs is stored and subsequently used to screen the ChemHopper™ library. Each fragment in the ChemHopper™ library is encoded into a latent vector using the VAE encoder, and cosine similarity is calculated between each fragment vector and the latent vector predicted by Slougs. Fragments with higher cosine similarity scores are considered hit fragments and selected for further examination.

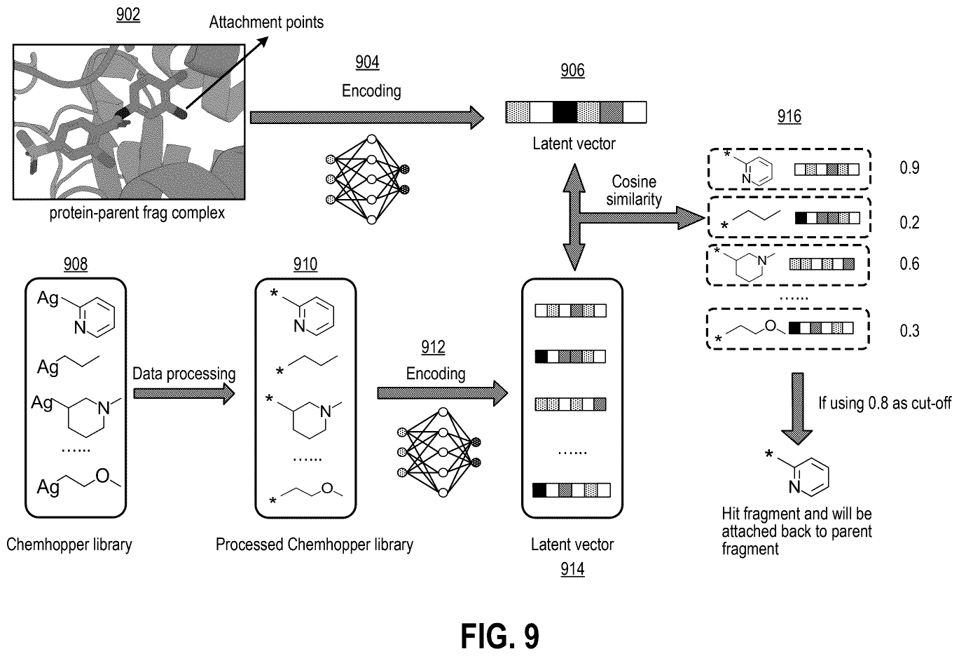

FIG. 9 is a schematic illustration for the Slougs workflow. 902 is a cartoon of a 3D protein structure file (.pdb) depicting the molecular interaction between the protein and the parent fragment. 904 is a schematic diagram of the E(3)-equivalent GNN which takes 902 as an input to produce the latent vector 906, corresponding to the interaction between the parent molecule and the protein of interest. 908 is a cartoon representing the ChemHopper™ library of peripheral fragments. 910 is a cartoon representing the product ChemHopper™ library using the parent fragment from 902. 912 is a schematic diagram of the VAE 904, which takes the molecules from 910 to produce a library of latent vectors 914 corresponding to each of those molecules. A cosine similarity analysis is performed between the parent latent vector 906 and the ChemHopper™ product latent vector library 914 to create a library of scored ChemHopper™ products 916. Products with a cosine similarity score greater than 0.8 are considered hit fragments.

The BindingNet v2 dataset contains 689,796 protein-ligand (PL) complexes extracted or modeled from PDB and ChEMBL databases. Due to computational resource constraints, a high-confidence subset was selected utilizing the hybrid score defined in Zhu et al (2025), resulting in 231, 978 complexes. Each complex was further processed following a similar protocol used in DeepFrag, Green et al. (2021), including the following steps:
a. residues within 6 Å of the ligand were extracted to serve as the protein input;
b. each ligand was split into multiple fragment pairs by iterating over all “cuttable bonds”;
c. dummy atoms were inserted at the cleavage points to mark the attachment sites on both fragments;
d. fragment pair where the smallest fragment was than 150 Daltons (Da) were filtered out;
e. and bond cleavage sites outside of 4 Å of the protein were filtered out, to ensure relevance of the binding pocket.

“Cuttable bonds” were defined as single bonds that are not part of a ring or aromatic system—i.e., bonds whose cleavage results in two separate fragments. For each ligand, all such bonds were iteratively cleaved to generate fragment pairs and saved as a tuple in the format of (protein_6A.pdb, original_ligand.sdf, parent_frag.sdf (big fragment), small_frag.sdf) for use in training stage. This exhaustive fragment strategy ensures comprehensive coverage of fragment space. While existing rule-based fragmentation methods such as RECAP and BRICS could account for synthetic accessibility of molecules, they typically yield less diverse fragments and fewer training pairs due to their reliance on predefined cleavage rules. Moreover, their limited flexibility can restrict applicability across diverse input complexes. In contrast, the iterative splitting approach generated a total of 1,795,968 fragment pairs, offering a rich and diverse dataset for training structure-directed generative models.

After fragment generation, the dataset was split into training and testing sets using a UMAP-based clustering approach, applied to Protein-Ligand Extended Connectivity (PLEC) interaction fingerprints extracted from each PL complex. Unlike conventional sequence similarity-based splitting, this method accounts for cases where proteins may have low overall sequence similarity but share similar binding site environments—an important consideration for structure-based learning tasks. The final split resulted in 1,793,350 complexes in the training set and 2,618 complexes in the test set.

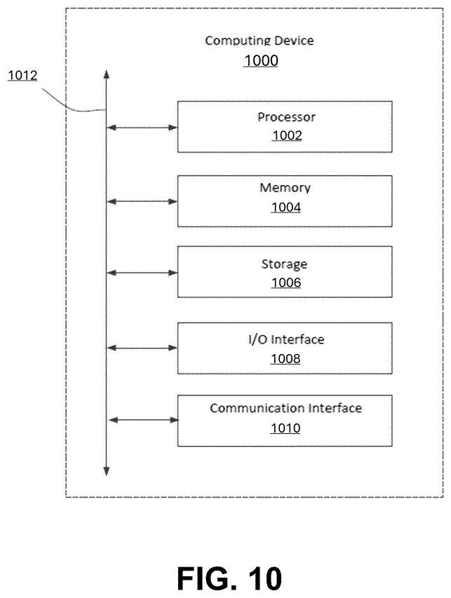

FIG. 10 is a block diagram illustrating an example computing device 1000. One or more computing devices such as the computing device 1000 may implement one or more processes described herein. For example, the computing device 1000 may comprise one or more of the engines of cheminformatics subsystem 204. As shown by FIG. 10, the computing device 1000 may comprise a processor 1002, a memory 1004, a storage device 1006, an I/O interface 1008, and a communication interface 1010, which may be communicatively coupled by way of a communication infrastructure 1012. The computing device 1000 may include fewer or more components than those shown in FIG. 10.

The processor 1002 may include hardware for executing instructions, such as those making up a computer application or system. In examples, to execute instructions for operating as described herein, the processor 1002 may retrieve (or fetch) the instructions from an internal register, an internal cache, the memory 1004, or the storage device 1006 and decode and execute the instructions. The memory 1004 may be a volatile or non-volatile memory used for storing data, metadata, computer-readable or machine-readable instructions, and/or programs for execution by the processor(s) for operating as described herein. The storage device 1006 may include storage, such as a hard disk, flash disk drive, or other digital storage device, for storing data or instructions for performing the methods.

The I/O interface 1008 may allow a user to provide input to, receive output from, and/or otherwise transfer data to and receive data from the computing device 1000. The I/O interface 1008 may include a mouse, a keypad or a keyboard, a touch screen, a camera, an optical scanner, network interface, modem, other known I/O devices or a combination of such I/O interfaces. The I/O interface 1008 may include one or more devices for presenting output to a user, including, but not limited to, a graphics engine, a display (e.g., a display screen), one or more output drivers (e.g., display drivers), one or more audio speakers, and one or more audio drivers. The I/O interface 1008 may be configured to provide graphical data to a display for presentation to a user. The graphical data may be representative of one or more graphical user interfaces and/or any other graphical content.

The communication interface 1010 may include hardware, software, or both. In any event, the communication interface 1010 may provide one or more interfaces for communication (such as, for example, packet-based communication) between the computing device 600 and one or more other computing devices and/or networks. The communication may be a wired or wireless communication. As an example, and not by way of limitation, the communication interface 1010 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1010 may facilitate communications with various types of wired or wireless networks. The communication interface 1010 may also facilitate communications using various communication protocols. The communication infrastructure 1012 may also include hardware, software, or both that couples components of the computing device 1000 to each other. For example, the communication interface 1010 may use one or more networks and/or protocols to enable a plurality of computing devices connected by a particular infrastructure to communicate with each other to perform one or more aspects of the processes.

In addition to what has been described herein, the methods and systems may also be implemented in a computer program(s), software, or firmware incorporated in one or more computer-readable media for execution by a computer(s) or processor(s), for example. Examples of computer-readable media include electronic signals (transmitted over wired or wireless connections) and tangible/non-transitory computer-readable storage media. Examples of tangible/non-transitory computer-readable storage media include, but are not limited to, a read only memory (ROM), a random-access memory (RAM), removable disks, and optical media such as CD-ROM disks, and digital versatile disks (DVDs).

What is claimed is:

1. At least one non-transitory computer-readable medium comprising instructions that, when executed by at least one processor, cause the at least one processor to:
receive a dataset of pharmaceutically active parent molecules;
identify a template constituent from a respective parent molecule at a cleavage site for each of the parent molecules of the dataset;
fragment, according to a first set of rules, each of the identified constituents;
sort, according to a second set of rules, each of the fragmented constituents into a backbone library or a peripheral library;
mutate, according to a third set of rules, each of the sorted backbone constituents and peripheral constituents;
systematically mark, according to a fourth set of rules, (a) each mutated backbone constituent with a dummy atom 1 as a parent attachment and a dummy atom 2 as a peripheral enumeration and (b) each mutated peripheral constituent with the dummy atom 2;
systematically combine respective marked backbone and peripheral constituents at any respective dummy atom 2, to generate a dataset of fragment modules; and
store the dataset as a searchable fragment module library.

2. The at least one non-transitory computer-readable medium of claim 1, wherein the first set of rules comprises cleaving all single bonds in any given constituent,
wherein cleaving results in a larger fragment and a smaller fragment, and
wherein the smaller fragment comprises at least one heavy atom and has a weight of <150 Da.

3. The at least one non-transitory computer-readable medium of claim 1, wherein the second set of rules comprises identifying each respective fragment as a backbone fragment unless (a) the number non-hydrogen atoms is ≤6 and (b) the cleavage site, which is the bond broken to create the fragment, is at a carbon atom, in which case the fragment is identified as a peripheral fragment.

4. The at least one non-transitory computer-readable medium of claim 1, wherein the third set of rules comprises:
(1), for any atoms of a given constituent, a mutation occurs only at a carbon;
(2), for any aromatic carbon of a given constituent, (a) if the number of hydrogens is 0, then no mutation occurs; (b) if the number of hydrogens is 1, then (i) the carbon is replaced by a nitrogen atom or a phosphorous atom or (ii) the hydrogen is be substituted with a halogen;
(3), for any aliphatic ring carbon, (a) if the number of hydrogens is 0, then no mutation occurs; (b) if the number of hydrogens is one, then (i) the carbon is replaced by a nitrogen atom or (ii) the hydrogen is substituted with a halogen; and (c) if the number of hydrogens is 2, then (i) the carbon is replaced by a nitrogen atom, (ii) the carbon and the two hydrogens is replaced by an oxygen atom or a sulfur atom, (iii) the carbon and two hydrogens is replaced by a carbonyl group; or (iv) one of the hydrogens is substituted with a halogen, and
(4), for any aliphatic chain carbon, (a) if the number of hydrogens is 0, then no mutation occurs; (b) if the number of hydrogens is one, then (i) the carbon is replaced by a nitrogen atom or (ii) the hydrogen is substituted with a halogen; and (c) if the number of hydrogens is 2 or 3, then (i) the carbon is replaced by a nitrogen atom, (ii) the carbon and two hydrogens is replaced by an oxygen atom or a sulfur atom, (iii) the carbon and two hydrogens is replaced by a carbonyl group; or (iv) one of the hydrogens is substituted with a halogen.

5. The at least one non-transitory computer-readable medium of claim 1, wherein the fourth set of rules comprises:
(1), for any given mutated backbone constituent, dummy atoms 1 and dummy atoms 2 are randomly and iteratively assigned to replace remaining hydrogens, and
(2), for any given mutated peripheral constituent, dummy atoms 2 are randomly and systematically assigned to replace remaining hydrogens to generate a pool of fragment modules.

6. The at least one non-transitory computer-readable medium of claim 5, wherein the fragment molecules are indexed by chemical structure using a simplified molecular input line entry system.

7. At least one non-transitory computer-readable medium comprising instructions that, when executed by at least one processor, cause the at least one processor to:
receive and store structural data for a pharmaceutically active parent molecule;
identify a search constituent at a cleavage site of the pharmaceutically active parent molecule;
access a searchable fragment module library to search structurally the library for complementary fragments to the search constituent,
wherein the searchable fragment module library is generated by
receiving a dataset of pharmaceutically active parent molecules;
identifying a template constituent from a respective parent molecule at a cleavage site for each of the patent molecules of the dataset;
fragmenting, according to a first set of rules, each of the identified constituents;
sort, according to a second set of rules, each of the fragmented constituents into a backbone library or a peripheral library;
mutating, according to a third set of rules, each of the sorted backbone and peripheral constituents;
systematically marking, according to a fourth set of rules, (a) each mutated backbone constituent with a dummy atom 1 as a parent attachment and a dummy atom 2 as a peripheral enumeration and (b) each mutated peripheral constituent with the dummy atom 2; and
systematically combine respective marked backbone constituents and peripheral constituents at any respective dummy atom 2, to generate a dataset of fragment modules; and
storing the dataset as the searchable fragment module library;
output a datafile of fragment module search results; and
systematically combine each fragment module of the datafile with the parent molecule between the cleavage site and a respective dummy atom 1 of any given fragment module to generate a dataset of iterative parent derivative molecules.

8. The at least one non-transitory computer-readable medium of claim 7, wherein the first set of rules comprises cleaving all single bonds in any given constituent,
wherein cleaving results in a larger fragment and a smaller fragment, and
wherein the smaller fragment comprises at least one heavy atom and has a weight of <150 Da.

9. The at least one non-transitory computer-readable medium of claim 7, wherein the second set of rules comprises identifying each respective fragment as a backbone fragment unless (a) the number non-hydrogen atoms is ≤6 and (b) the cleavage site, which is the bond broken to create the fragment, is at a carbon atom, in which case the fragment is identified as a peripheral fragment.

10. The at least one non-transitory computer-readable medium of claim 7, wherein the third set of rules comprises:
(1), for any atoms of a given constituent, a mutation occurs only at a carbon;
(2), for any aromatic carbon of a given constituent, (a) if the number of hydrogens is 0, then no mutation occurs; (b) if the number of hydrogens is 1, then (i) the carbon is replaced by a nitrogen atom or a phosphorous atom or (ii) the hydrogen is be substituted with a halogen;
(3), for any aliphatic ring carbon, (a) if the number of hydrogens is 0, then no mutation occurs; (b) if the number of hydrogens is one, then (i) the carbon is replaced by a nitrogen atom or (ii) the hydrogen is substituted with a halogen; and (c) if the number of hydrogens is 2, then (i) the carbon is replaced by a nitrogen atom, (ii) the carbon and the two hydrogens is replaced by an oxygen atom or a sulfur atom, (iii) the carbon and two hydrogens is replaced by a carbonyl group; or (iv) one of the hydrogens is substituted with a halogen, and
(4), for any aliphatic chain carbon, (a) if the number of hydrogens is 0, then no mutation occurs; (b) if the number of hydrogens is one, then (i) the carbon is replaced by a nitrogen atom or (ii) the hydrogen is substituted with a halogen; and (c) if the number of hydrogens is 2 or 3, then (i) the carbon is replaced by a nitrogen atom, (ii) the carbon and two hydrogens is replaced by an oxygen atom or a sulfur atom, (iii) the carbon and two hydrogens is replaced by a carbonyl group; or (iv) one of the hydrogens is substituted with a halogen.

11. The at least one non-transitory computer-readable medium of claim 7, wherein the fourth set of rules comprises:
(1), for any given mutated backbone constituent, dummy atoms 1 and dummy atoms 2 are randomly and iteratively assigned to replace remaining hydrogens, and
(2), for any given mutated peripheral constituent, dummy atoms 2 are randomly and systematically assigned to replace remaining hydrogens to generate a pool of fragment modules.

12. The at least one non-transitory computer-readable medium of claim 7, wherein the dataset of iterative parent derivative molecules is output as a datafile and filtered to generate a subset of drug candidate molecules.

13. At least one non-transitory computer-readable medium comprising instructions that, when executed by at least one processor, cause the at least one processor to:
receive a dataset of pharmaceutically active parent molecules;
identify a template constituent from a respective parent molecule at a cleavage site for each of the parent molecules of the dataset;
fragment, according to a first set of rules, each of the identified constituents;
storing each of the fragmented constituents into a backbone library;
identify, according to a second set of rules, a set of backbone constituents and peripheral constituents;
store a copy set of peripheral constituents into a peripheral library such that the backbone library and peripheral library contain respective original and copy sets of the peripheral constituents;
mutate, according to a third set of rules, the stored constituents in each of the backbone and peripheral libraries;
systematically mark, according to a fourth set of rules, (a) each mutated constituent in the backbone library with a dummy atom 1 as a parent attachment and a dummy atom 2 as a peripheral enumeration and (b) each mutated constituent in the peripheral library with the dummy atom 2;
systematically combine respective constituents in the backbone and peripheral libraries at any respective dummy atom 2, to generate a dataset of fragment modules; and
store the dataset as a searchable fragment module library.

14. The at least one non-transitory computer-readable medium of claim 13, wherein the first set of rules comprises cleaving all single bonds in any given constituent,
wherein cleaving results in a larger fragment and a smaller fragment, and
wherein the smaller fragment comprises at least one heavy atom and has a weight of <150 Da.

15. The at least one non-transitory computer-readable medium of claim 13, wherein the second set of rules comprises identifying each respective fragment as a backbone fragment unless (a) the number non-hydrogen atoms is ≤6 and (b) the cleavage site, which is the bond broken to create the fragment, is at a carbon atom, in which case the fragment is identified as a peripheral fragment.

16. The at least one non-transitory computer-readable medium of claim 13, wherein the third set of rules comprises:
(1), for any atoms of a given constituent, a mutation occurs only at a carbon;
(2), for any aromatic carbon of a given constituent, (a) if the number of hydrogens is 0, then no mutation occurs; (b) if the number of hydrogens is 1, then (i) the carbon is replaced by a nitrogen atom or a phosphorous atom or (ii) the hydrogen is be substituted with a halogen;
(3), for any aliphatic ring carbon, (a) if the number of hydrogens is 0, then no mutation occurs; (b) if the number of hydrogens is one, then (i) the carbon is replaced by a nitrogen atom or (ii) the hydrogen is substituted with a halogen; and (c) if the number of hydrogens is 2, then (i) the carbon is replaced by a nitrogen atom, (ii) the carbon and the two hydrogens is replaced by an oxygen atom or a sulfur atom, (iii) the carbon and two hydrogens is replaced by a carbonyl group; or (iv) one of the hydrogens is substituted with a halogen, and
(4), for any aliphatic chain carbon, (a) if the number of hydrogens is 0, then no mutation occurs; (b) if the number of hydrogens is one, then (i) the carbon is replaced by a nitrogen atom or (ii) the hydrogen is substituted with a halogen; and (c) if the number of hydrogens is 2 or 3, then (i) the carbon is replaced by a nitrogen atom, (ii) the carbon and two hydrogens is replaced by an oxygen atom or a sulfur atom, (iii) the carbon and two hydrogens is replaced by a carbonyl group; or (iv) one of the hydrogens is substituted with a halogen.

17. The at least one non-transitory computer-readable medium of claim 13, wherein the fourth set of rules comprises:
(1), for any given mutated constituent in the backbone library, dummy atoms 1 and dummy atoms 2 are randomly and iteratively assigned to replace remaining hydrogens, and
(2), for any given mutated constituent in the peripheral library, dummy atoms 2 are randomly and systematically assigned to replace remaining hydrogens to generate a pool of fragment modules.

18. The at least one non-transitory computer-readable medium of claim 17, wherein the fragment molecules are indexed by chemical structure using a simplified molecular input line entry system.
